(19) United States
(12) Patent Application Publication
(10) Pub. No.: US 2016/0302970 A1
(43) Pub. Date: Oct. 20, 2016

(54) Probes for Use In Ophthalmic and Vitreoretinal Surgery

(71) Applicant: CYGNUS LP, Roswell, GA (US)

(72) Inventor: Fouad Mansour, Sandy Springs, GA (US)

(21) Appl. No.: 15/194,998

(22) Filed: Jun. 28, 2016

Related U.S. Application Data
(63) Continuation-in-part of application No. 15/160,254, filed on May 20, 2016.
Application No. 15/160,254 is a continuation-in-part of application No. 13/270,028, filed on Oct. 10, 2011, now Pat. No. 9,370,447.

(51) Int. Cl. A61F 9/008

(52) U.S. Cl. CPC A61F 9/00821; A61F 2009/00874; A61F 2009/00863

(57) ABSTRACT

A probe for endo-ocular photocoagulation procedures provides straight and curved tip configurations. A flexible tubular material, pre-formed with a radius of curvature, allows the tip to be inserted through a trocar cannula and to resume its pre-formed shape for use during a surgical procedure. Alternatively, a tubular material is pre-formed with a radius of curvature, and is stiffened by use of a preferably stainless steel tube. Inside the tubular material is a distal end of at least one optical fiber, inserted such that its tip is coterminous with the tubular material. A fixed tube surrounds the optical fiber. The tubular material is configured to freely move relative to the fixed tube and along the axis of the assembly, acting to straighten the tubular material and optical fiber members as it moves forward via a sliding member that is associated with the hand piece. Illumination energy, laser energy, or both may be supplied to the targeted surgical site.

### CROSS-REFERENCE TO RELATED APPLICATIONS

The application is a continuation-in-part of and claims priority to U.S. patent application Ser. No. 15/160,254, filed May 20, 2016, which is a continuation-in-part of and claims priority to U.S. patent application Ser. No. 13/270,028, filed Oct. 10, 2011 and now U.S. Pat. No. 9,370,447 issued on Jun. 21, 2016, the entire contents of which are incorporated herein by reference.

### TECHNICAL FIELD

The present disclosure relates generally to ocular surgery devices; and, more particularly, to probes for use in ophthalmic and vitreoretinal surgery. In some embodiments, a fixed curvature laser probe tip is provided that flexes when penetrating a trocar cannula; in other embodiments, a variable curvature laser probe tip is provided that is operable between straight and curved tip configurations.

### BACKGROUND

A common treatment often utilized in ophthalmic and vitreoretinal surgery is that of directing laser energy to a surgical site, the targeted surgical site typically being proximate a patient's retina and the surrounding vitreous. Such a surgery is called an endo-ocular photocoagulation procedure, and may be indicated for reattachment of a detached retina, for cauterization of a ruptured blood vessel, for repair of a surgical wound, for removal of defective tissue or vitreous material, and the like.

In order to conduct the endo-ocular photocoagulation procedure, the surgeon must utilize a microsurgical laser probe to deliver the laser energy to the surgical site. The microsurgical laser probe typically comprises a handle with a small cylindrical sleeve, or tip, projecting from the distal end of the handle. An optical fiber element is connected at the proximal end to a laser source, and the fiber is carried through the microsurgical laser probe and into the cylindrical sleeve. The optical fiber element is positioned adjacent the distal end of the cylindrical sleeve in order to effectively deliver laser energy to the intended surgical site.

Prior to beginning the surgery, the surgeon must ascertain and select the appropriate size and type of microsurgical laser probe tip to be used. Currently, microsurgical laser probe tips are available in three predominant sizes: 20 gauge (0.0360 inches), 23 gauge (0.0255 inches), and 25 gauge (0.0205 inches). For smaller gauge probe tips, the use of an appropriately sized trocar cannula is indicated. The trocar cannula is used to pierce the patient's ocular tissue and, thereafter, to provide a passageway for the insertion and support of the probe tip, to prevent the probe tip from bending at the point of entry into the eye, to reduce tearing of the ocular tissue at the insertion point, and to act as a guide channel for the probe tip into the eye. Accordingly, 23 and 25 gauge tips are nearly always used in association with trocar cannulae; whereas, because of relatively larger size and stiffness, 20 gauge tips do not typically require use of trocar cannulae.

When using a 20 gauge probe tip, the surgeon pierces or punctures the ocular tissue in a selected location, inserts the probe tip to the appropriate depth, angle, and position, and begins the endo-ocular photocoagulation procedure. When using either a 23 or 25 gauge probe tip, the surgeon first pierces or punctures the ocular tissue in a selected location with a trocar cannula and positions it on the eye. Thereafter, the 23 or 25 gauge probe tip is passed through the trocar cannula and into the eye to the appropriate depth, angle, and position. Thereafter, the endo-ocular photocoagulation procedure may be conducted.

It is instructive to note that, in order to be most surgically effective, the laser energy should be delivered as nearly perpendicular to the targeted surgical area as possible. Due to positioning of the microsurgical laser probe tip, and the positioning of any associated trocar cannula that may be utilized by the surgeon during the procedure to direct the microsurgical laser probe into the eye, it is most often the case that the surgical site is either inaccessible or located disadvantageously for proper application of laser energy.

In order to solve this problem, curved tips have been introduced for use in association with above-described microsurgical laser probes. Use of such curved tips is advantageous in comparison to use of a straight tip in better orienting the probe tip adjacent the surgical site without need to withdraw and reposition the laser probe, and without the associated secondary punctures of the eye; and, further, in providing for a greater range of coverage inside the eye. Disadvantageously, such curved tips are of fixed curvature and, depending upon the relative diameters of the tip and the associated trocar cannula, sometimes cannot be inserted through straight trocar cannulae. Furthermore, if a different curvature is required in order to properly target the surgical site, the instrument must be withdrawn, and the entire probe must be replaced with one having a tip of appropriate curvature, and the initial wound site re-intruded. Such process is, of course, less than optimal for both patient and surgeon.

To overcome these disadvantages, others have introduced adjustable, directional laser probes that are capable of being adjustably manipulated toward a target surgical site; thereby, seeking to avoid some of the above-referenced disadvantages, while seeking to better direct laser energy to the targeted surgical area. Some such devices are discussed below, and their referenced disclosures are incorporated herein by reference.

U.S. Pat. No. 6,572,608 to Lee et al. provides a probe with a handle and a tubular sleeve. The distal portion of the tubular sleeve has an optical fiber projecting therefrom that can be caused to bend relative to the sleeve by manual manipulation of a mechanism on the probe handle, as the optical fiber is enclosed within a pre-formed curved memory material, such as Nitinol. Disadvantageously, whenever exterior parts of a device move, there is a risk that foreign materials can become lodged within or between those exterior moving parts. Because the Lee et al. design utilizes moving, exterior mechanical parts which move relative to one another within the eye, there is an attendant risk that tissue may become snagged or tangled therebetween. Additionally, when deploying the tip outwardly, the surgeon must carefully ascertain where the tip is located relative to the intended target surgical site in order to prevent puncturing the back of the eye. The surgeon must continually keep a finger positioned on the extension/retraction mechanism to control deployment and retraction of the probe tip, while simultaneously controlling insertion and withdrawal of the probe tip. In other words, the surgeon must undertake a two-step, iterative targeting procedure wherein he/she must withdraw or insert the probe tip, adjust the tip curvature, and repeat until the targeted area can be appropriately accessed. This is a complex, non-intuitive surgical manipulation of the probe instrument, with possible damage resulting to the retina or other ocular tissue.

U.S. Pat. No. 6,984,230 to Scheller et al., a continuation-in-part of U.S. Pat. No. 6,572,608 to Lee et al., further provides that a tubular member carrying the optical fiber may be deployed outwardly from the sleeve. Disadvantageously, and as with U.S. Pat. No. 6,572,608 to Lee et al., such design utilizes moving, exterior mechanical parts which move relative to one another within the eye, with an attendant risk that tissue may become snagged or tangled therebetween. Additionally, and as with Lee et al., when deploying the tip outwardly, the surgeon must carefully ascertain where the tip is located relative to the intended target surgical site in order to prevent puncturing the back of the eye. The surgeon must continually keep a finger positioned on the extension/retraction mechanism to control deployment and retraction of the probe tip, while simultaneously controlling insertion and withdrawal of the probe tip. In other words, the surgeon must undertake a two-step, iterative targeting procedure wherein he/she must withdraw or insert the probe tip, adjust the tip curvature, and repeat until the targeted area can be appropriately accessed. This is a complex, non-intuitive surgical manipulation of the probe instrument, with possible damage resulting to the retina or other ocular tissue.

In order to overcome such disadvantages, U.S. Pat. No. 7,766,904 to McGowen, Sr. et al. provides a laser probe capable of functioning in both straight and curved forms. The probe includes an elongated hand piece and rigid cannula affixed thereto to prevent relative translational movement between them. A pre-curved optical fiber inside a memory material, such as Nitinol, extends through the hand piece and cannula, and a slidable button is affixed to the optical fiber through use of a cooperating rigid sleeve. The relative motion of the button with respect to the handle is tied, both visually and physically, to the relative extension, and the resulting curvature, of the optical fiber, with the intended result being avoidance or minimization of damage to the retina or other ocular tissue. Unfortunately, many of the same disadvantages may be seen; to wit, such design utilizes mechanical parts which move relative to one another within the eye, with an attendant risk that tissue may become snagged or tangled therebetween. Further, when deploying the tip outwardly, the surgeon must still guess, or follow additional procedures to ascertain, where the tip is located relative to the intended target surgical site. The surgeon must continually keep a finger positioned on the extension/retraction mechanism to control deployment and retraction of the tip, while simultaneously controlling insertion and withdrawal of the probe tip (the two-step, iterative targeting procedure described above). This results in a complex, non-intuitive surgical manipulation of the instrument, with possible damage resulting to the retina or other ocular tissue.

United States Patent Application Publication Number 2010/0004642 A1 by Lumpkin proposes a surgical laser probe wherein a hand piece carries an optical fiber through and into a stainless steel tube, the distal end of which carries a length of polyimide bendable tube. The optical fiber is coterminous with the free end of the polyimide tube. A slidable element is installed within a slot in the hand piece and secured to a length of pre-curved Nitinol wire. As the slideable wire element is moved toward the distal end of the hand piece, the Nitinol wire is advanced into the free end of the polyimide tube, increasingly bending the tube and optical fiber away from the longitudinal axis of the stainless steel tube and hand piece. Disadvantageously, this design bends the optical fiber tip about a single point; thus, having a relatively small local radius of curvature, resulting in a relatively large, straight-line offset of the tip. Accordingly, the surgeon may not be able to reach the targeted surgical site due to insufficient curvature of the tip. Furthermore, a small local radius of curvature can, in some cases, reduce or interfere with laser transmission if the radius is below the value recommended by the manufacturer of the optical fiber.

As was discussed above, probe tips having a fixed curvature sometimes cannot be inserted through straight trocar cannulae, depending upon the relative diameters of the tip and the associated trocar cannula. For example, many trocar cannulae are manufactured according to proprietary or customized specifications, such that a trocar cannula manufactured by one company will not be fully compatible with surgical solutions provided by another company. Accordingly, a probe tip providing fixed curvature may be compatible with one model or size of trocar cannula, but not with another. An exemplary solution is proposed in U.S. Pat. No. 7,909,816 to Buzawa, which provides a rigid probe for use with a rigid cannula, the probe having an outside diameter smaller than the inside diameter of the cannula, the probe having one or more sections comprising radii of curvature selected to ensure passage of the probe through the length of the cannula without interference. This is seen to be disadvantageous not only in probe tip manufacture, but also in adding to the complexity of surgical probe entry into and removal from the cannula, and in manipulation and movement of the probe tip during a procedure.

Yet additionally, in most surgeries, one or more additional surgical tool(s), such as a surgical-site illumination instrument, may be needed. Such additional surgical instruments may require additional penetrations into the eye tissue, in which case the recovery time for the patient may be increased, and the risk of complications may likewise be increased.

In an attempt to reduce the need for such additional instruments, particularly those requiring a separate intrusion into the eye, laser energy surgical probes have been designed to deliver both laser energy for treatment and illumination energy, such as for visualization of the targeted surgical site, using a single probe. Thus, only a single penetration into the eye may be required for visualization of the targeted surgical area, as well as for delivery of laser energy to the area to accomplish the surgery.

As such, it is clear that there is an unmet need for an ophthalmic surgical device capable of delivering illumination energy and/or laser energy for treatment of a patient's eye via a single wound site, while maintaining a small diameter probe for reducing unwanted injury to the eye.

Accordingly, what is still needed is an adjustable, directional laser probe for endo-ocular photocoagulation procedures providing, in some embodiments, a fixed curvature laser probe tip that flexes when penetrating a trocar cannula; and in other embodiments, a variable curvature laser probe tip that is operable between straight and curved tip configurations, and that may deliver one or both of laser energy for treatment and illumination energy for visualization of the targeted surgical site, all using a single probe, and all while eliminating or reducing the above-referenced disadvantages in accordance with the detailed disclosure of the inventive subject matter set forth hereinbelow. It is to the provision of such probes that the present disclosure is directed.

### SUMMARY

The present disclosure is a laser probe for endo-ocular photocoagulation procedures which provides, in some embodiments, a fixed curvature laser probe tip that flexes when penetrating a trocar cannula; and in other embodiments, a variable curvature laser probe tip that is operable between straight and curved tip configurations. In an exemplary embodiment, a hand held fiber optic assembly connects one or more of a light and a laser source via connectors at a proximal end, with the distal, delivery end for use inside the eye when held by a surgeon. The laser energy may be used for endo-ocular photocoagulation procedures involving the retina, surrounding tissue, and vitreous. Illumination energy may, primarily or optionally, be supplied to illuminate the targeted surgical site.

More particularly, in some tip embodiments, an engineered, flexible, plastic tubular material is pre-formed with a section comprising a desired radius of curvature. The curved portion of the tip is sufficiently flexible to straighten sufficiently to pass through a conventional trocar cannula and into the eye, whereafter it resumes its pre-formed, curved shape for use during a surgical procedure.

In other tip embodiments, an engineered, tubular material is pre-formed with a section comprising a desired radius of curvature, and is stiffened internally or externally by use of a preferably stainless steel material. As an example, the tubular material may comprise a shape memory material, particularly a shape memory polymer. Inside the plastic tubular material is a distal end of an optical fiber, inserted such that its tip is coterminous with the plastic tubular material. A second or other material, such as stainless steel, Nitinol, or the like, surrounds the optical fiber and is free to move along the axis of the assembly, acting to straighten the plastic and optical fiber members as it moves forward via a sliding member, such as a button, that is associated with the hand piece.

Through use of a device according to some embodiments of the present disclosure, the surgeon has the ability to utilize a fixed-curve probe tip that will pass easily through a straight, conventional trocar cannula, whereafter the original probe tip shape is resumed for the surgical procedure.

Through use of a device according to other embodiments of the present disclosure, the surgeon has the ability to change the location for energy delivery by changing the angle at the tip by use of a sliding mechanism that acts gradually to straighten a preformed curve at the tip of the probe until a desired angle is achieved. The relative axial movement between the straightening member and the pre-formed curvature at the tip determines the angle of delivery. Advantageously, such a design gives the surgeon the ability to cover a large area compared to a fixed angle tip, while avoiding most of the problems noted in the prior art.

These and other features and advantages of the present disclosure will become more apparent to those of ordinary skilled in the art after reading the following Detailed Description of Illustrative Embodiments of the disclosure and the Claims in light of the accompanying drawing Figures.

### BRIEF DESCRIPTION OF THE DRAWINGS

Accordingly, the present disclosure will be understood best through consideration of, and with reference to, the following drawing Figures, viewed in conjunction with the Detailed Description of Illustrative Embodiments of the disclosure referring thereto, in which like reference numbers throughout the various Figures designate like structure, and in which:

FIG. 1A is a partial cut-away side view of one embodiment of a flexible tip laser probe for endo-ocular photocoagulation procedures according to the present disclosure;

FIG. 1B is an enlarged, partial cut-away side view of an embodiment of a flexible tip laser probe for endo-ocular photocoagulation procedures according to FIG. 1A of the present disclosure;

FIG. 1C is a partial cut-away side view of another embodiment of a flexible tip laser probe for endo-ocular photocoagulation procedures according to the present disclosure;

FIG. 1D is a partial cut-away side view of another embodiment of a flexible tip laser probe for endo-ocular photocoagulation procedures according to the present disclosure;

FIG. 2A is a partial cut-away side view of a directional tip laser probe for endo-ocular photocoagulation procedures according to the present disclosure;

FIG. 2B is a partial cut-away side view of a directional tip laser probe for endo-ocular photocoagulation procedures according to FIG. 2A of the present disclosure, illustrating movement of the tip thereof between a straight and a curved configuration;

FIG. 3A is partial cut-away side view of one embodiment of a directional tip laser probe for endo-ocular photocoagulation procedures according to FIG. 2A of the present disclosure, illustrating the tip thereof in a curved configuration;

FIG. 3B is a partial cut-away side view of one embodiment of a directional tip laser probe for endo-ocular photocoagulation procedures according to FIG. 2B of the present disclosure, illustrating the tip thereof in a straight configuration;

FIG. 4A is partial cut-away side view of another embodiment of a directional tip laser probe for endo-ocular photocoagulation procedures according to the present disclosure, illustrating the tip thereof in a curved configuration;

FIG. 4B is a partial cut-away side view of another embodiment of a directional tip laser probe for endo-ocular photocoagulation procedures according to FIG. 4A of the present disclosure, illustrating the tip thereof in a straight configuration;

FIG. 5A is partial cut-away side view of another embodiment of a directional tip laser probe for endo-ocular photocoagulation procedures according to the present disclosure, illustrating the tip thereof in a curved configuration;

FIG. 5B is a partial cut-away side view of another embodiment of a directional tip laser probe for endo-ocular photocoagulation procedures according to FIG. 5A of the present disclosure, illustrating the tip thereof in a straight configuration;

FIG. 6A is a partial cut-away side view of another embodiment of a directional tip laser probe for endo-ocular photocoagulation procedures according to the present disclosure;

FIG. 6B is a partial cut-away side view of another embodiment of a directional tip laser probe for endo-ocular photocoagulation procedures according to FIG. 6A of the present disclosure, illustrating the tip thereof in a curved configuration;

FIG. 6C is a partial cut-away side view of another embodiment of a directional tip laser probe for endo-ocular photocoagulation procedures according to FIG. 6A of the present disclosure, illustrating the tip thereof in a straight configuration;

FIG. 7A is a partial cut-away side view of another embodiment of a directional tip laser probe for endo-ocular photocoagulation procedures according to FIG. 6A of the present disclosure, illustrating the tip thereof in a curved configuration;

FIG. 7B is a partial cut-away side view of another embodiment of a directional tip laser probe for endo-ocular photocoagulation procedures according to FIG. 6A of the present disclosure, illustrating the tip thereof in a straight configuration;

FIG. 8A is a partial cut-away side view of another embodiment of a directional tip laser probe for endo-ocular photocoagulation procedures according to the present disclosure, illustrating the tip thereof in a curved configuration;

FIG. 8B is a partial cut-away side view of another embodiment of a directional tip laser probe for endo-ocular photocoagulation procedures according to FIG. 8A of the present disclosure, illustrating the tip thereof in a straight configuration;

FIG. 9A is a partial cut-away side view of another embodiment of a directional tip laser probe for endo-ocular photocoagulation procedures according to the present disclosure, illustrating the tip thereof in a curved configuration; and

FIG. 9B is a partial cut-away side view of another embodiment of a directional tip laser probe for endo-ocular photocoagulation procedures according to FIG. 9A of the present disclosure, illustrating the tip thereof in a straight configuration.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION OF ILLUSTRATIVE EMBODIMENTS

In describing preferred embodiments of the present disclosure illustrated in the figures, specific terminology is employed for the sake of clarity. The disclosure, however, is not intended to be limited to the specific terminology so selected, and it is to be understood that each specific element includes all technical equivalents that operate in a similar manner to accomplish a similar purpose.

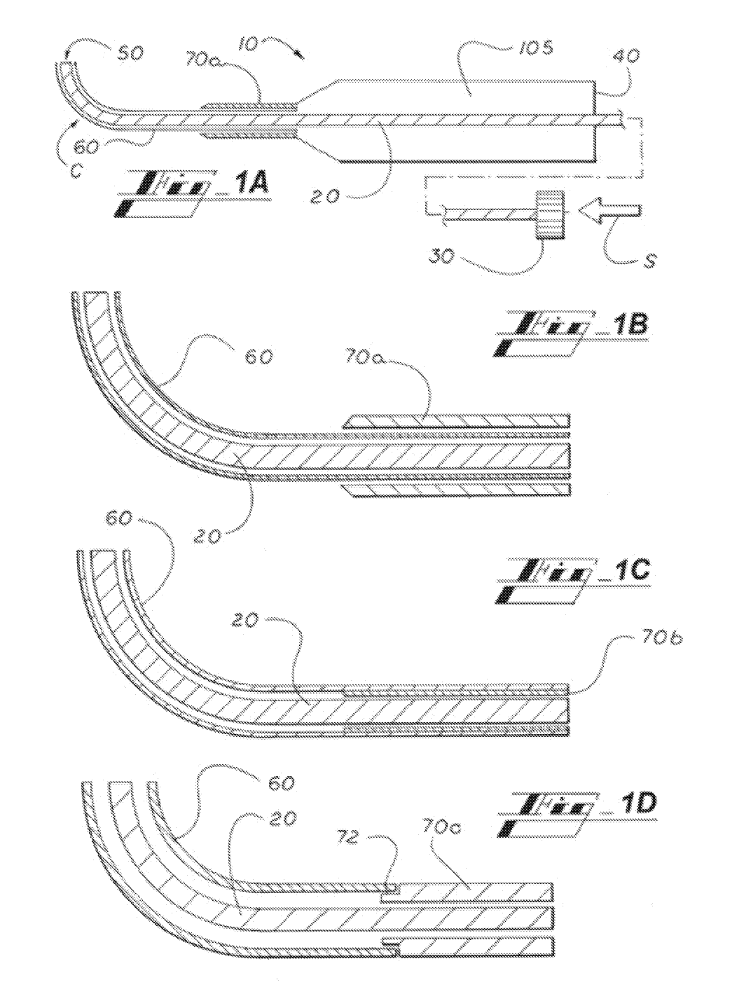

In a form of the present disclosure chosen for purposes of illustration, exemplary embodiments of which are illustrated in FIGS. 1A, 1B, 1C, and 1D, a flexible tip laser probe for endo-ocular photocoagulation procedures is shown. Hand held fiber optic assembly 10 connects one or more of a light and a laser source S through one or more optical fibers 20 via one or more connector 30 disposed from proximal end 40, with the distal, delivery end 50 of such one or more optical fibers 20 for use inside the eye when held by a surgeon at hand piece 105. The laser energy may be used for endo-ocular photocoagulation procedures involving the retina, surrounding tissue, and vitreous. Illumination energy may be supplied to illuminate the targeted surgical site. Exemplary of such a combined laser and illumination energy delivery device is Applicant's U.S. patent application Ser. No. 11/934,761, filed on Nov. 3, 2007, now U.S. Pat. No. 8,647,333, the disclosure of which is hereby incorporated by reference. In other embodiments, one or more dedicated illumination optical fiber may run parallel to one or more dedicated laser energy optical fiber and connect to respective illumination and laser sources.

In typical embodiments, flexible tip laser probe 10 is provided in 23 and 25 gauge sizes; however, it may also be provided in 20 gauge or other sizes. It is noted that, in accordance with said reference disclosure, flexible tip laser probe 10 for endo-ocular photocoagulation procedures may deliver one or both of laser energy for treatment and illumination energy for visualization of the targeted surgical site.

An engineered, preferably plastic, flexible, tubular material 60 is pre-formed with a radius of curvature C. Tubular material 60 is preferably formed of polyether ether ketone (PEEK), which is an organic polymer thermoplastic material. In other embodiments, however, tubular material 60 may be formed using any material capable of maintaining a pre-formed curvature, flexing to a straightened form, and then returning to its pre-formed curvature. As one example of such a material, tubular material 60 may be formed of a shape memory material, particularly a shape memory polymer (SMP), including physically crosslinked SMPs, chemically crosslinked SMPs, and electroactive SMPs. Suitable examples of a physically crosslinked SNIP include polyurethane (including those with ionic or mesogenic components made by a pre-polymer method), block copolymers (e.g., block copolymer of polyethylene terephthalate (PET), block copolymer of polyethyleneoxide (PEO), block copolymers containing polystyrene and/or poly(1,4-butadiene), an ABA triblock copolymer made from poly(2-methyl-2-oxazoline) and/or polytetrahydrofuran), linear amorphphous polynorbornene, or organic-inorganic hybrid polymers consisting of polynorbornene units that are partially substituted by polyhedral oligosilsesquioxane (POSS). Suitable examples of a chemically crosslinked SMP include crosslinked polyurethane or a PEO-based crosslinked material, such as PEO-PET. Suitable examples of an electroactive SMP include material made, at least in part, from carbon nanotubes, short carbon fibers (SCF), carbon black, metallic nickel powder, or surface-modified super-paramagnetic nanoparticles (e.g., an oligo (e-capolactone)dimethacrylate/butyl acrylate composite with between 2 and 12% magnetite nanoparticles), nickel fibers, or nickel-hybrid fibers. Other suitable materials from which tubular material 60 may be formed include polyethylene, polypropylene, or nylon.

In the embodiment shown in FIGS. 1A and 1B, tubular material 60 is overlayed by, or inserted into, a rigid portion 70a of hand piece 105. Rigid portion 70a serves to support and stabilize the tip assembly during use. Inside plastic tubular material 60 is disposed a distal end of optical fiber 20, inserted such that its tip is coterminous with the plastic tubular material 60.

Rigid portion 70a preferably is selected to meet one of the industry-standard outside diameters; to wit, 20, 23, or 25 gauges. So configured, the present disclosure may be utilized, if desired or required by a surgeon, with a standard size and configuration of trocar cannula. Importantly, as is well-known in the art, larger tip sizes, such as 20 gauge, do not require use of trocar cannulae, but may require suturing of the intrusion site(s). Use of larger tip sizes may also require longer patient healing times. On the other hand, smaller tip sizes, such as 23 or 25 gauge, typically do require use of trocar cannulae, but do not typically require suturing. Use of smaller tip sizes may also shorten patient healing times. Accordingly, it is noted that smaller tip sizes, such as 23 or 25 gauge, are preferred for increasing the rate of a patient's post-surgical recovery, for reducing trauma, and for increasing post-surgical comfort; however, it is contemplated that any appropriate tip size(s) may be utilized.

Specifically, tubular material 60 of the present disclosure is flexible, rather than rigid, and does not move with respect to hand piece 105. Similarly, optical fiber 20 of the present disclosure is fixed with respect to hand piece 105 and tubular material 60. This design reduces the potential for puncture or other wounds to retinal or surrounding tissue.

Distinctively, the curved portion of the tip is flexible enough to straighten sufficiently to pass through a conventional trocar cannula and into the eye; whereafter, it resumes its pre-formed, curved shape for use during a surgical procedure. With this design, no special considerations are required of the surgeon with regard to either tip or trocar cannula sizes, other than the conventional surgical choice of compatible tip and trocar cannula size.

FIG. 1C depicts an alternative embodiment of flexible tip laser probe for endo-ocular photocoagulation procedures. All particulars of design, construction, and use, and all other attendant considerations, are as set forth above with regard to the embodiment of FIGS. 1A and 1B, except insofar as will now be discussed. In the embodiment of FIG. 1C, tubular material 60 is underlayed by, or pressed over, a rigid portion 70b of hand piece 105. Rigid portion 70b serves to support and stabilize the tip assembly during use. In this embodiment, tubular material 60 preferably is selected to meet one of the industry-standard outside diameters; to wit, 20, 23, or 25 gauges.

FIG. 1D depicts an alternative embodiment of flexible tip laser probe for endo-ocular photocoagulation procedures. All particulars of design, construction, and use, and all other attendant considerations, are as set forth above with regard to the embodiment of FIGS. 1A and 1B, except insofar as will now be discussed. In the embodiment of FIG. 1D, tubular material 60 is underlayed by, or pressed over, a stepped or shouldered end 72 of rigid portion 70c of hand piece 105. Stepped or shouldered end 72 is of reduced outer diameter, disposed to be concentrically joined with plastic tubular material 60. One of ordinary skill in the art will recognize, however, that stepped or shouldered end 72 may, alternatively, be of reduced inside diameter, disposed to be concentrically joined with plastic tubular material 60. Rigid portion 70c serves to support and stabilize the tip assembly during use. In this embodiment, tubular material 60 and rigid portion 70c preferably are selected to meet one of the industry-standard outside diameters; to wit, 20, 23, or 25 gauges.

Other methods and constructions for forming rigid portion 70 are fully contemplated hereby.

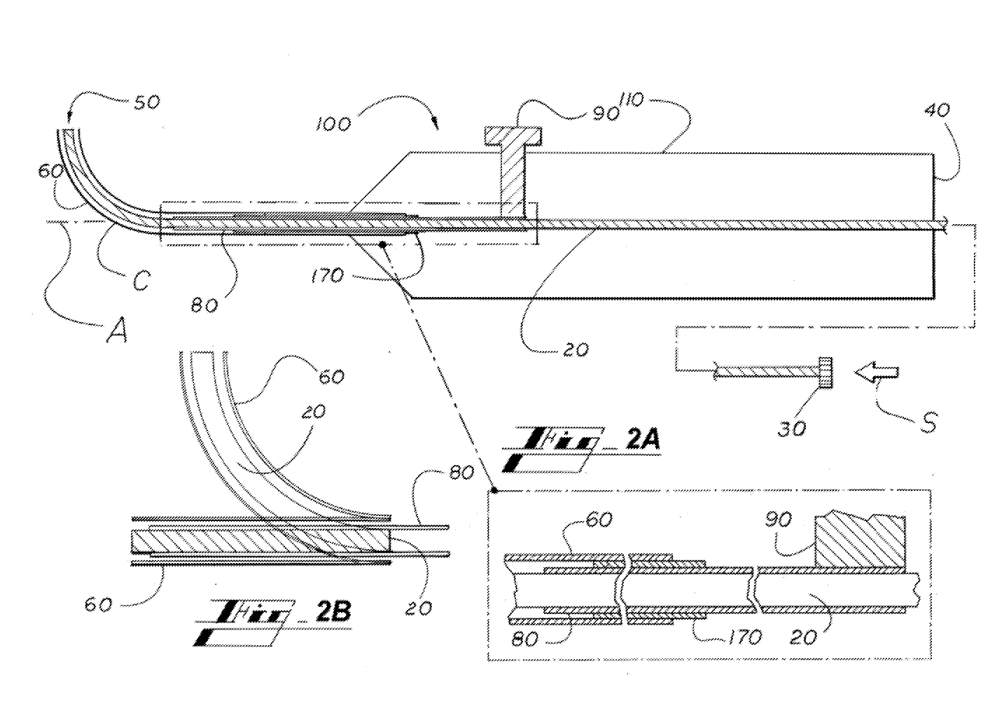

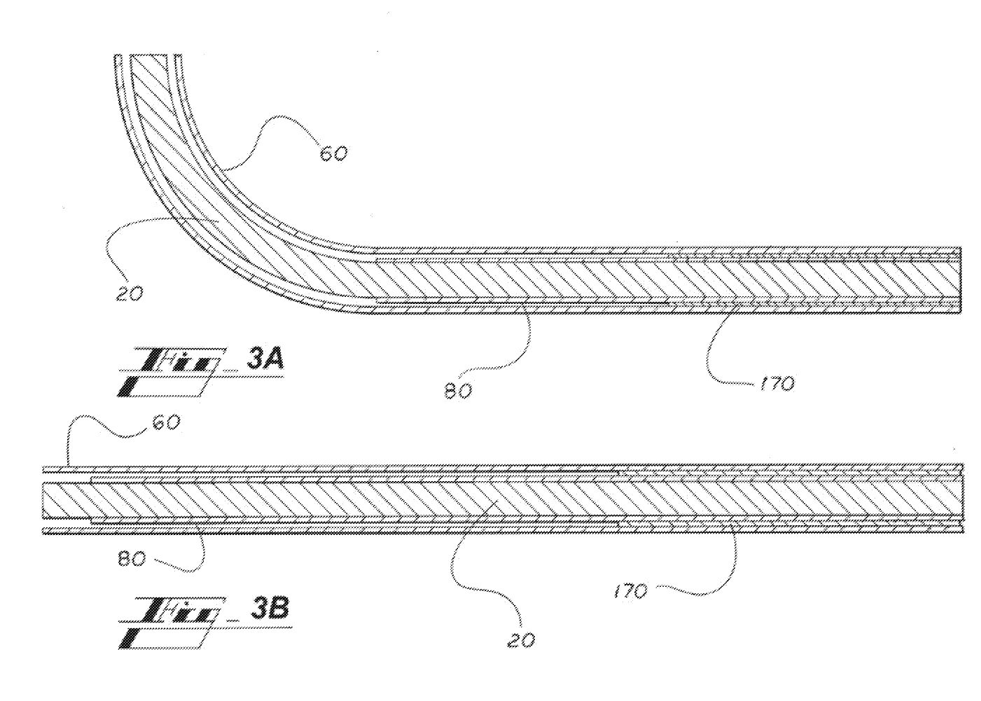

Turning now to a form of the present disclosure chosen for purposes of illustration, exemplary embodiments of which are illustrated in FIGS. 2A, 2B, 3A, and 3B, a directional tip laser probe for endo-ocular photocoagulation procedures is shown. Hand held fiber optic assembly 100 connects one or more of a light and a laser source S through optical fiber 20 via one or more connector 30 disposed from proximal end 40, with the distal, delivery end 50 for use inside the eye when held by a surgeon at hand piece 110. The laser energy may be used for endo-ocular photocoagulation procedures involving the retina, surrounding tissue, and vitreous. Illumination energy may be supplied to illuminate the targeted surgical site, as discussed above with regard to a combined laser and illumination energy delivery device set forth within Applicant's U.S. patent application Ser. No. 11/934,761, filed on Nov. 3, 2007, now U.S. Pat. No. 8,647,333, the disclosure of which has been incorporated by reference. In other embodiments, one or more dedicated illumination optical fiber may run parallel to one or more dedicated laser energy optical fiber and connect to respective illumination and laser sources.

In typical embodiments, flexible tip laser probe 100 is provided in 20, 23, and 25 gauge sizes; however, it may also be provided in other sizes. It is noted that, in accordance with the reference disclosure discussed above, flexible tip laser probe 100 for endo-ocular photocoagulation procedures may deliver one or both of laser energy for treatment and illumination energy for visualization of the targeted surgical site.

An engineered, preferably plastic tubular material 60 is pre-formed with a radius of curvature C, and is stiffened internally in this embodiment, or externally in other embodiments, by use of a preferably stainless steel, Nitinol, or other suitable rigid material, tube 170. Tubular material 60 may be formed of polyether ether ketone (PEEK), an organic polymer thermoplastic material described above; however, one of ordinary skill in the art will recognize that a suitable substitute material would be capable of holding a pre-formed curvature, flexing to a straightened form, and then returning approximately to its pre-formed curvature, as described above in further detail. Accordingly, in some embodiments, Nitinol may serve as an appropriate substitute material. Inside plastic tubular material 60 is disposed a distal end of optical fiber 20, inserted such that its tip is coterminous with the plastic tubular material. Tube 80, formed of a second or other suitable rigid material, such as stainless steel, Nitinol, or the like, surrounds optical fiber 20 and is free to move along longitudinal axis A of the assembly, acting to straighten plastic tubular material 60 and optical fiber 20 as it moves forward, e.g., toward distal, delivery end 50, via sliding member 90, such as a button, that is associated with hand piece 110. It should be apparent that the construction of the device of the present disclosure should ensure that tube 80 does not pass the tip of optical fiber 20 when tube 80 is fully deployed.

Tubular material 60 preferably is selected to meet one of the industry-standard outside diameters; to wit, 20, 23, or 25 gauges. So configured, the present disclosure may be utilized, if desired or required by a surgeon, with a standard size and configuration of trocar cannula. Importantly, as is well-known in the art, larger tip sizes, such as 20 gauge, do not require use of trocar cannulae, but may require suturing of the intrusion site(s). Use of larger tip sizes may also require longer patient healing times. On the other hand, smaller tip sizes, such as 23 or 25 gauge, typically do require use of trocar cannulae, but do not typically require suturing. Use of smaller tip sizes may also shorten patient healing times. Accordingly, it is noted that smaller tip sizes, such as 23 or 25 gauge, are preferred for increasing the rate of a patient's post-surgical recovery, for reducing trauma, and for increasing post-surgical comfort; however, it is contemplated that any appropriate tip size(s) may be utilized.

In some embodiments, stainless steel tube 80 may be replaced with a wire of appropriate dimensions and stiffness. In some embodiments, stainless steel tube 80 may be the moving part; or, alternatively, the rest of the assembly could be allowed to move; or, still further alternatively, a combination of both could be effectuated. As will be apparent to one of ordinary skill in the art, what is important is the relative motion interoperably established amongst the defined elements.

It may be observed that, advantageously with the present disclosure, optical fiber 20 and tubular material 60 do not move with respect to longitudinal axis A, as distinguished from some exemplary prior art devices. By extending and retracting tube 80 via sliding member 90, such as a button, that is associated with hand piece 110, curvature C may be manipulated between a straightened configuration, best seen with reference to FIGS. 2B, 3B, 4B, 5B, or curved configuration, best seen with reference to FIGS. 2A, 3A, 4A, 5A. This allows for intuitive surgical manipulation, as has been described above.

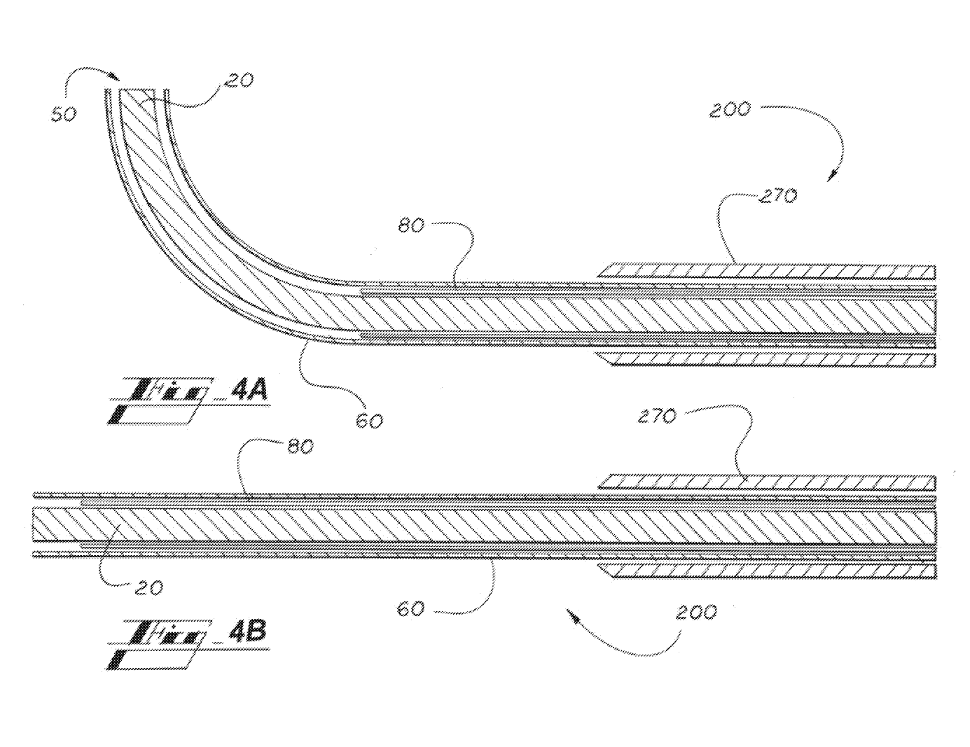

Turning now to FIGS. 4A and 4B, an alternative embodiment 200 of the present disclosure is shown. Except as noted, construction of alternative embodiment 200 is equivalent to the embodiment of FIGS. 2A, 2B, 3A, 3B. In alternative embodiment 200, reinforcing tube 270 is provided in order to strengthen and stiffen the construction of distal end 50. In this embodiment, reinforcing tube 270 preferably is selected to meet one of the industry-standard outside diameters; to wit, 20, 23, or 25 gauges. Reinforcing tube 270 is preferably formed of stainless steel or Nitinol and is fit over tubular material 60. As in previous embodiments, FIG. 4A illustrates stainless steel tube 80 fully retracted and tip curved. FIG. 4B illustrates stainless steel tube 80 fully deployed and tip straightened.

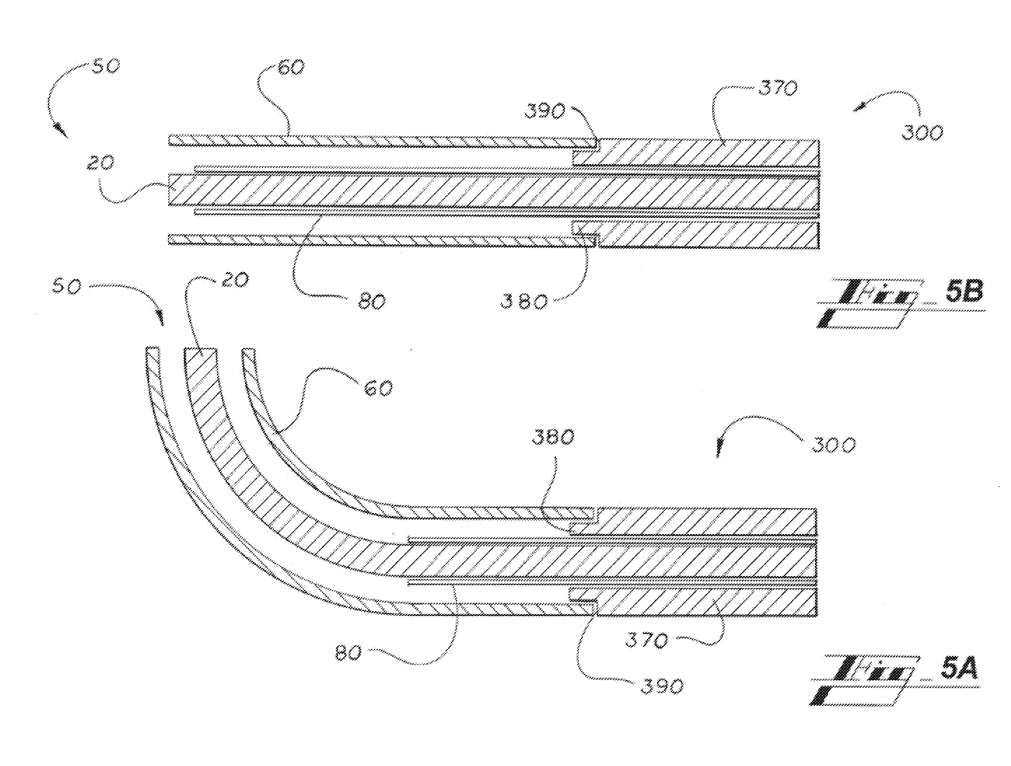

Turning now to FIGS. 5A and 5B, an alternative embodiment 300 of the present disclosure is shown. Except as noted, construction of alternative embodiment 300 is equivalent to the embodiment of FIGS. 2A, 2B, 3A, 3B. In alternative embodiment 300, reinforcing tube 370 is provided in order to strengthen and stiffen the construction of distal end 50. In this embodiment, reinforcing tube 370 preferably is selected to meet one of the industry-standard outside diameters; to wit, 20, 23, or 25 gauges. Reinforcing tube 370 is preferably formed of stainless steel or Nitinol. In some embodiments, a distal end of reinforcing tube 370 is provided with reduced outer diameter 380, disposed to be concentrically joined with plastic tubular material 60 at joint 390. Other methods and constructions for forming joint 390 are fully contemplated hereby. As in previous embodiments, FIG. 5A illustrates stainless steel tube 80 fully retracted and tip curved. FIG. 5B illustrates stainless steel tube 80 fully deployed and tip straightened.

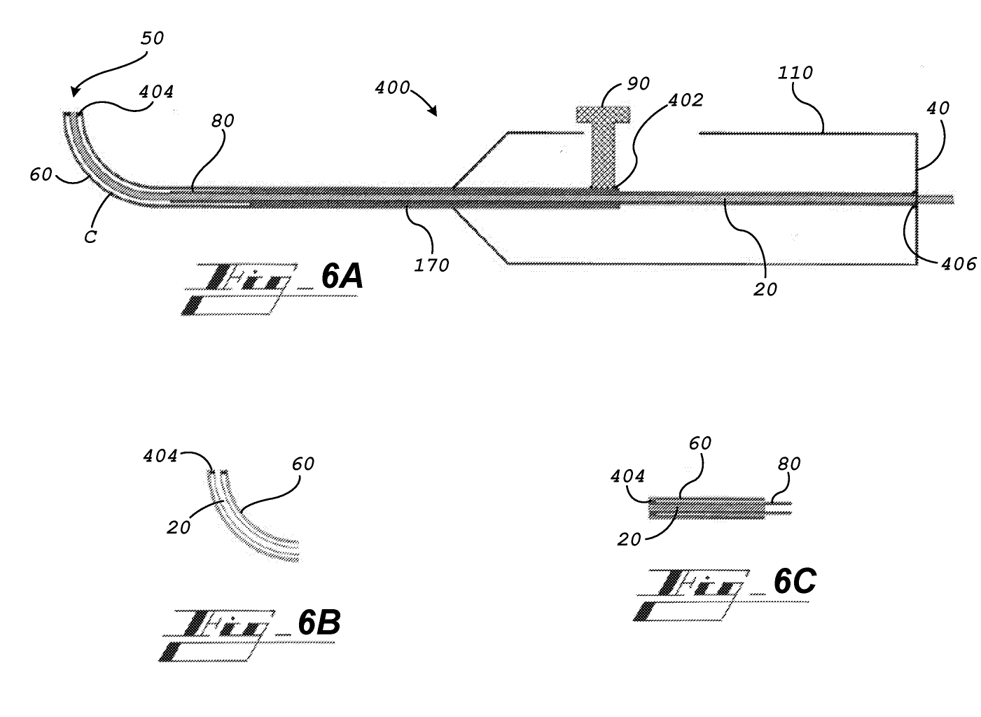

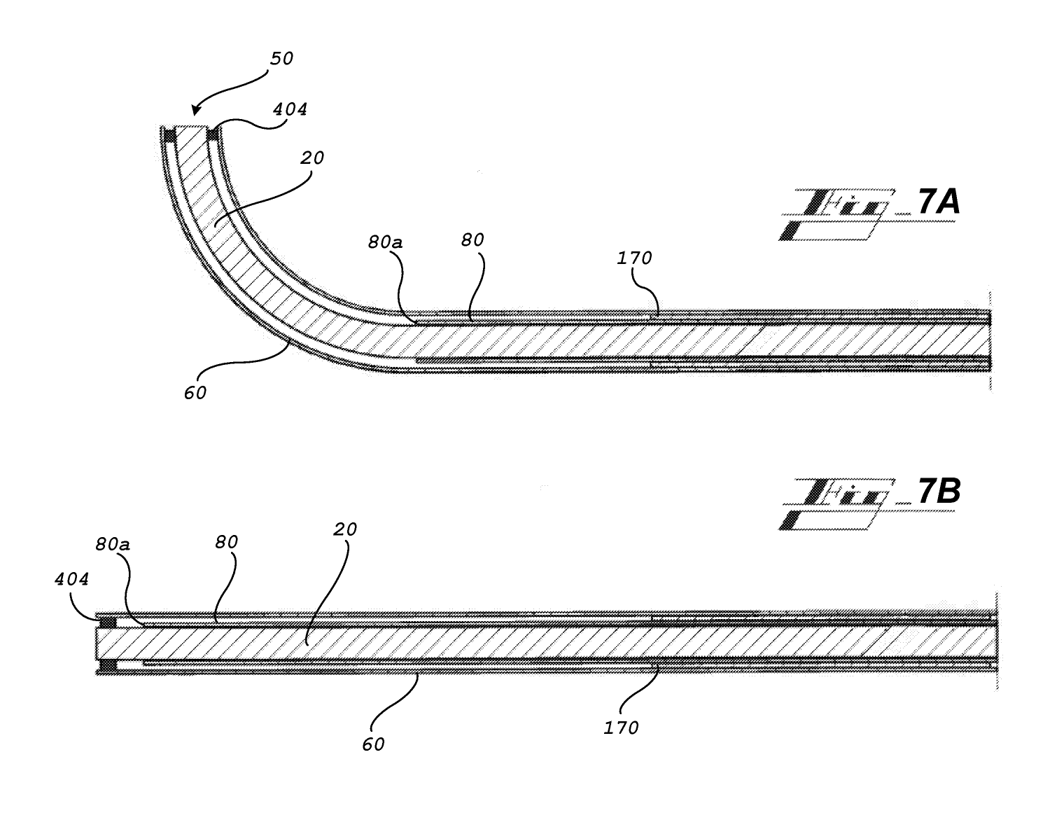

Turning now to FIGS. 6A, 6B, 6C, 7A, and 7B, an alternative embodiment 400 of the present disclosure is shown in which tube 80 is fixed relative to hand piece 110 and the optical fiber 20 and tubular material 60 are movable relative to hand piece 110. Except as noted, construction of alternative embodiment 400 is equivalent to the embodiment of FIGS. 2A, 2B, 3A, and 3B. In alternative embodiment 400, sliding member 90 is fixed to tubular material 60 via attachment point 402. Optical fiber 20 is fixed to tubular material 60 such that optical fiber 20 moves longitudinally in conjunction with tubular material 60 when the movement of tubular material 60 is effectuated. Optical fiber 20 and tubular material 60 are depicted in alternative embodiment 400 as attached via attachment point 404 near distal, delivery end 50. Yet in some aspects, attachment point 404 may be located elsewhere along the longitudinal lengths of optical fiber 20 and tubular material 60. At least a portion of tubular material 60 is movably secured by hand piece 110 such that tubular material 60 and hand piece 110 are movable relative to one another along their respective longitudinal axes. Tube 170, composed of stainless steel, Nitinol, or other suitably rigid material, is disposed within at least a portion of the straight length of tubular material 60 to stiffen and support said portion of tubular material 60. In one aspect, tube 170 may be fixed to tubular material 60, while in other aspects tube 170 may instead be fixed to tube 80.

Further in alternative embodiment 400, tube 80, which surrounds optical fiber 20 and acts to straighten tubular material 60 and optical fiber 20, is fixed to hand piece 110 via attachment point 406. In the embodiment shown in FIG. 6A, attachment point 406 is located near proximal end 40. In other aspects, attachment point 406 may be located at any point at which hand piece 110 and tube 80 contact. By fixing tube 80 to hand piece 110, tubular material 60 is movable relative to both tube 80 and hand piece 110, thereby causing tube 80 to extend and retract within curvature C of tubular material 60.

In use, curvature C may be manipulated from a straightened configuration, shown in FIGS. 6C and 7B, to a curved configuration, shown in FIGS. 6A, 6B, and 7A, by hand piece 110 (and therefore also tube 80) being held stationary while tubular material 60 is extended, such as via sliding member 90, away from hand piece 110 and terminal end 80a of tube 80 to deploy the curved tip of tubular material 60 beyond terminal end 80a and, in general, tube 80. The process may be reversed to return curvature C to the straightened configuration. Similarly, curvature C may be manipulated from a straightened configuration to a curved configuration by the tubular material 60 being held stationary, such as by an operator gripping sliding member 90, while hand piece 110 (and therefore also tube 80) is moved backwards towards proximal end 40, thereby retracting tube 80 from tubular material 60 and allowing tubular material 60 to return to its curved shape. Again, this process may be reversed to return curvature C to the straightened configuration.

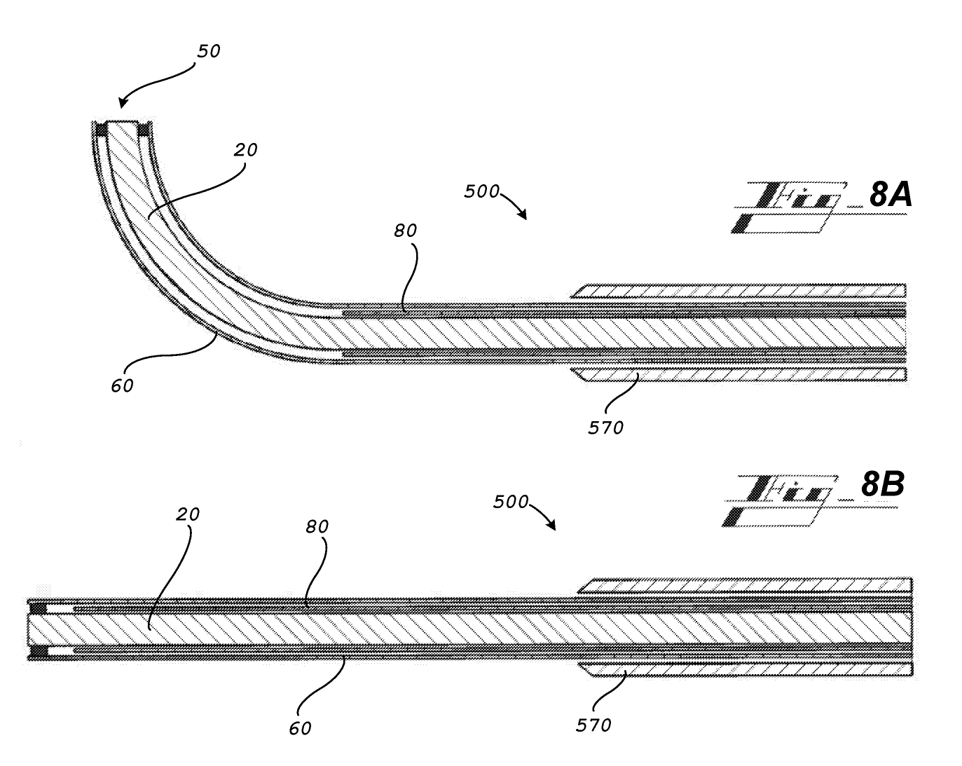

Turning now to FIGS. 8A and 8B, an alternative embodiment 500 of the present disclosure is shown. Except as noted, construction of alternative embodiment 500 is equivalent to the embodiment of FIGS. 6A, 6B, 6C, 7A, and 7B. In alternative embodiment 500, reinforcing tube 570 is concentrically provided around the outside diameter of tubular material 60. Like reinforcing tube 170 discussed in relation to FIGS. 4A and 4B, reinforcing tube 570 provides support to at least a portion of tubular material 60 and may be sized according to one of the industry-standard outside diameters (e.g., 20, 23, or 25 gauge). In alternative embodiment 500, reinforcing tube 570 is fixed to tubular material 60 (and therefore unfixed to hand piece 110) so that reinforcing tube 570 moves in conjunction with tubular material 60. In such an embodiment, sliding member 190 may be fixed to either tubular material 60 or reinforcing tube 170. Alternatively, in an aspect, reinforcing tube 570 may be fixed to hand piece 110 and unfixed to tubular material 60, allowing free longitudinal movement between reinforcing tube 570 and tubular material 60. Accordingly, sliding member 190 may be fixed to tubular material 60. As in previous embodiments, FIG. 8A illustrates tubular material 60 extended beyond tube 80 in a curved configuration. FIG. 8B illustrates tubular material 60 retracted over tube 80 such that tube 80 causes tubular material 60 to assume a straight configuration.

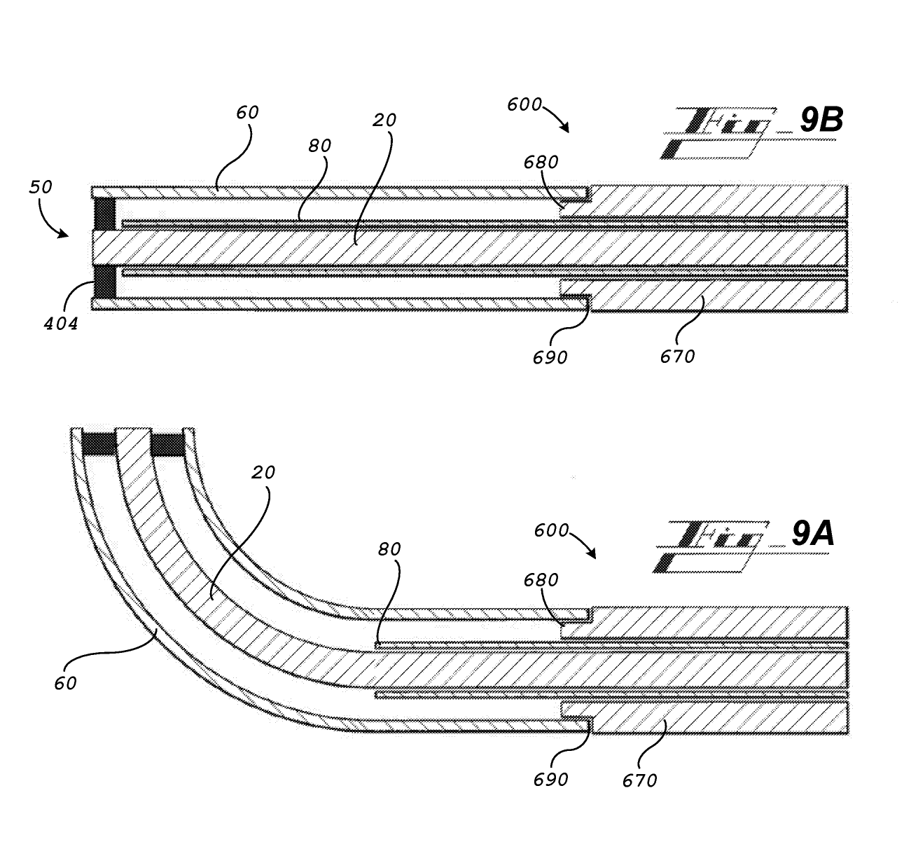

Turning now to FIGS. 9A and 9B, an alternative embodiment 600 of the present disclosure is shown. Except as noted, construction of alternative embodiment 600 is equivalent to the embodiment of FIGS. 6A, 6B, 6C, 7A, and 7B. In alternative embodiment 600, reinforcing tube 670, which is similar to reinforcing tube 370 discussed in relation to FIGS. 5A and 5B, is provided to strengthen and stiffen distal, delivery end 50, including at least a portion of tubular material 60. Reinforcing tube 670 may be sized according to one of the industry-standard outside diameters (e.g., 20, 23, or 25 gauge) and formed of stainless steel, Nitinol, or other suitably rigid material. A distal end of reinforcing tube 370 is provided with reduced outer diameter 680, which concentrically joins with tubular material 60 at joint 690. Since tubular material 60 is so attached to reinforcing tube 670, sliding member 190 may be attached in this embodiment to reinforcing tube 670 to effectuate the movement of tubular material 60 relative to tube 80. As in previous embodiments, FIG. 9A illustrates tubular material 60 extended beyond tube 80 in a curved configuration. FIG. 9B illustrates tubular material 60 retracted over tube 80 such that tube 80 causes tubular material 60 to assume a straight configuration.

It will be appreciated that while the tubular material 80 is generally referred to and depicted in FIGS. 6A-C, 7A-B, 8A-B, and 9A-B (as well as the remaining figures) as a tube, the disclosure is not so limited. The tubular material 80 may alternatively be embodied as a wire, rod, shaft, or other straight structure.

As should now be apparent, through use of a directional tip laser probe for endo-ocular photocoagulation procedures according to the present disclosure, the surgeon has the ability to change the location for energy delivery by changing the angle at the tip of the probe by use of a sliding mechanism that acts gradually to straighten a preformed curve at the tip of the probe until a desired angle is achieved. The relative axial movement between the straightening member and the pre-formed curvature at the tip determines the angle of energy delivery. Advantageously, such a design gives the surgeon the ability to cover a large area compared to a fixed angle tip, while avoiding most of the problems noted in the prior art.

Specifically, tubular material 60 of the present disclosure is flexible, rather than rigid, and does not move with respect to hand piece 110. Similarly, optical fiber 20 of the present disclosure is fixed with respect to hand piece 110 and tubular material 60. This design reduces the potential for puncture or other wounds to retinal or surrounding tissue. It should be apparent to one of ordinary skill in the art, however, that the present disclosure advantageously provides for relative axial movement between the straightening member and the pre-formed curvature at the tip to determine the angle of delivery. Accordingly, one could provide an embodiment wherein tube 80 is fixed, and wherein optical fiber 20 and tubular material 60 are allowed to move. All such embodiments are fully contemplated hereby.

Further tubular material 60, and by virtue thereof, optical fiber 20 are pre-bent and internally straightened. This design, accordingly, presents no externally moving parts into the eye; thereby, reducing the potential for snagging or tangling of ocular tissue.

Still further, the design of the present disclosure provides the surgeon with a more intuitive manipulation of the probe than was possible with exemplary prior art devices, such as those of U.S. Pat. Nos. 6,984,230 and/or 7,766,904, as the fiber optic tip is showing all of the time, and there is no guessing where it will deploy. Further the curvature C of the device approximately follows the curvature of the eye.

Even further, the design of the present disclosure provides better suited and more aggressive angles than exemplary prior art devices, such as that of U.S. Patent Application 2010/0004642, due to the geometry of the device and a more efficient use of space. The present device offers surgeons a truly curved tip, which is advantageous over other designs which bend principally about a single point or which have a relatively small radius of curvature.

In considering other and further alternative embodiments of the present disclosure, the following observations should become apparent to one of ordinary skill in the art. For example, it should become apparent that the straightening member is not restricted to a tubular construction. Such straightening member could equivalently take the form of a wire, a rod, or the like; however, a tubular construction is preferred for the reason that it offers enhanced stiffness in tight geometries than do the alternatives.

Although tip curvature has been shown in an “upward” configuration, it could be at any angle, dependent upon surgical requirements.

The preferred material for tubular material 60 has been established as PEEK, due to its unique capability to be shaped with heat and to thereafter retain its shape. In alternative embodiments, however, tubular material 60 could be of any material or materials, including layers of materials, that can be straightened by a sliding member on the inside and then return to a pre-formed curvature, as described above in greater detail.

Some embodiments of the present disclosure may include members that are not circular in cross-section, and which may be, for example, oval shapes, elongated shapes, and the like.

Some embodiments of the present disclosure may include a straightening member that may not, itself, be straight. In such embodiments, the straightening member may be curved in a fashion so that the combined curvature with the tip would achieve a straight tip configuration. This may be the case, for example, where the straightening member must overcome the forces attendant a curvature of the PEEK (or other material) member.

In some embodiments, the tip of the straightening member may be shaped in a fashion so as to facilitate movement inside the curved PEEK (or other material) member and not cut into the optical fiber. For example, the end of the straightening member may be disposed at an angle, with the longer portion close to the side of the PEEK material (or other material) side with the larger radius, and the shorter portion close to the side of the PEEK material (or other material) with the shorter radius.

Having thus described exemplary embodiments of the present disclosure, it should be noted by those ordinarily skilled in the art that the within disclosures are exemplary only and that various other alternatives, adaptations, and modifications may be made within the scope and spirit of the present disclosure. Accordingly, the present disclosure is not limited to the specific embodiments as illustrated herein, but is only limited by the following claims.

What is claimed:

1. A surgical probe for endo-ocular photocoagulation, the surgical probe comprising:
a. a hand piece;
b. at least one optical fiber associated with the hand piece for carrying light energy from a light energy source to a distal end of said optical fiber;
c. a flexible, pre-curved tip comprising a straight portion and a proximal portion, said straight and proximal portions associated with the hand piece at a distal end thereof, said pre-curved tip carrying said distal end of said optical fiber, said pre-curved tip comprising a shape memory polymer, and wherein said flexible, pre-curved tip has an outer diameter of 20 gauge or less;
d. a rigid jacket associated with said proximal portion to support and stabilize said flexible, pre-curved tip;
e. said distal end of said optical fiber being coterminous with a distal end of said pre-curved tip;
f. a rigid straightening member fixed relative to said hand piece and operably associated with said optical fiber and said pre-curved tip, said straightening member at least partially disposed within said straight portion of said pre-curved tip;
g. said pre-curved tip operable to extend and retract along a longitudinal axis of the surgical probe; and
wherein, in a first mode of operation, said pre-curved tip is configured to extend away from said hand piece and said straightening member, acting to allow said pre-curved tip and optical fiber to resume a curved form, and wherein, in a second mode of operation, said pre-curved tip is configured to retract toward said hand piece and cause said straightening member to be positioned toward said distal end of said pre-curved tip, acting to straighten said distal end of said pre-curved tip as said pre-curved tip retracts.

2. The surgical probe of claim 1, wherein said rigid jacket is fixedly attached to said pre-curved tip.

3. The surgical probe of claim 1, wherein said rigid jacket is fixedly attached to said hand piece.

4. The surgical probe of claim 1, wherein the shape memory polymer comprises polyether ether ketone (PEEK).

5. The surgical probe of claim 1, wherein the shape memory polymer comprises at least one of: polyurethane, a block copolymer of polyethylene terephthalate (PET), a block copolymer of polyethyleneoxide (PEO), a block copolymers containing polystyrene, a block copolymer containing poly(1,4-butadiene), an ABA triblock copolymer made from poly(2-methyl-2-oxazoline), an ABA triblock copolymer made from polytetrahydrofuran, linear amorphphous polynorbornene, or an organic-inorganic hybrid polymer consisting of polynorbornene units that are partially substituted by polyhedral oligosilsesquioxane (POSS).

6. The surgical probe of claim 1, wherein the shape memory polymer comprises at least one of: crosslinked polyurethane or a polyethyleneoxide (PEO)-based crosslinked material.

7. The surgical probe of claim 1, wherein the shape memory polymer comprises a material comprising at least one of: carbon nanotubes, short carbon fibers (SCF), carbon black, metallic nickel powder, surface-modified super-paramagnetic nanoparticles, nickel fibers, or nickel-hybrid fibers.

8. The surgical probe of claim 1, wherein the shape memory polymer comprises at least one of: polyethylene, polypropylene, or nylon.

9. The surgical probe of claim 1, wherein said pre-curved tip is associated with a sliding member that is associated with the hand piece, said sliding member operable to effectuate the first mode of operation or the second mode of operation.

10. The surgical probe of claim 9, wherein said sliding member is fixedly attached to said pre-curved tip.

11. The surgical probe of claim 1, wherein said straightening member is tubular.

12. The surgical probe of claim 1, wherein said straightening member comprises a wire.

13. A probe for ophthalmic and vitreoretinal surgery, the probe comprising:
a. a hand piece;
b. at least one optical fiber associated with the hand piece for carrying laser energy from a laser energy source to a distal end of said optical fiber;
c. a flexible, pre-curved tip comprising a straight portion and a proximal portion, said straight and proximal portions associated with the hand piece at a distal end thereof, said pre-curved tip carrying said distal end of said optical fibers, said pre-curved tip comprising a shape memory polymer, and wherein said flexible, pre-curved tip has an outer diameter of 20 gauge or less;
d. a rigid jacket associated with said proximal portion to support and stabilize said flexible, pre-curved tip;
e. said distal ends of said optical fibers being coterminous with a distal end of said pre-curved tip;
f. a rigid straightening member fixedly attached to said hand piece;
g. said straightening member disposed in association with said optical fibers and within said pre-curved tip;
h. said pre-curved tip operable to extend and retract along a longitudinal axis of the probe; and
wherein, in a first mode of operation, said pre-curved tip is configured to extend away from said hand piece and said straightening member, acting to allow said pre-curved tip and optical fiber to resume a curved form, and wherein, in a second mode of operation, said pre-curved tip is configured to retract toward said hand piece and cause said straightening member to be positioned toward said distal end of said pre-curved tip, acting to straighten said distal end of said pre-curved tip as said pre-curved tip retracts.

14. The probe of claim 11, wherein said rigid jacket is fixedly attached to said pre-curved tip.

15. The probe of claim 11, wherein said rigid jacket is fixedly attached to said hand piece.

16. The probe of claim 11, wherein the shape memory polymer comprises polyether ether ketone (PEEK).

17. The probe of claim 11, wherein the shape memory polymer comprises at least one of: polyurethane, a block copolymer of polyethylene terephthalate (PET), a block copolymer of polyethyleneoxide (PEO), a block copolymers containing polystyrene, a block copolymer containing poly(1,4-butadiene), an ABA triblock copolymer made from poly(2-methyl-2-oxazoline), an ABA triblock copolymer made from polytetrahydrofuran, linear amorphphous polynorbomene, or an organic-inorganic hybrid polymer consisting of polynorbomene units that are partially substituted by polyhedral oligosilsesquioxane (POSS).

18. The probe of claim 11, wherein the shape memory polymer comprises at least one of: crosslinked polyurethane or a polyethyleneoxide (PEO)-based crosslinked material.

19. The probe of claim 11, wherein the shape memory polymer comprises a material comprising at least one of: carbon nanotubes, short carbon fibers (SCF), carbon black, metallic nickel powder, surface-modified super-paramagnetic nanoparticles, nickel fibers, or nickel-hybrid fibers.

20. The probe of claim 11, wherein the shape memory polymer comprises at least one of: polyethylene, polypropylene, or nylon.

21. The probe of claim 11, wherein said pre-curved tip is associated with a sliding member that is associated with the hand piece, said sliding member operable to effectuate the first mode of operation or the second mode of operation.

22. The probe of claim 19, wherein said sliding member is fixedly attached with said pre-curved tip.

23. The surgical probe of claim 13, wherein said straightening member is tubular.

24. The surgical probe of claim 13, wherein said straightening member comprises a wire.
